(19) United States
(12) Patent Application Publication
(10) Pub. No.: US 2004/0110271 A1
(43) Pub. Date: Jun. 10, 2004

(54) Organic waste treatment system

(76) Inventor: Gregory S. Patterson, Maple Grove, MN (US)

Correspondence Address: SHERRILL LAW OFFICES, 4756 BANNING AVE, SUITE 212, WHITE BEAR LAKE, MN, 55110-3205, US

(21) Appl. No.: 10/315,751

(22) Filed: Dec. 10, 2002

(51) Int. Cl. C12N 1/20; C05F 11/08

(52) U.S. Cl. 435/262; 71/8; 435/252.4

(57) ABSTRACT

A process for treating waste comprising the steps of (i) adding a first innoculum comprising sulfide-utilizing bacteria to a sulfide-rich organic waste to produce a lowered-sulfide waste, (ii) adding a second innoculum comprising organic digesting bacteria to the lowered-sulfide waste to produce a digested waste, and (iii) adding to the digested waste at least one of (a) a third innoculum comprising bacteria capable of converting a nitrogen-containing compound to N₂ to produce a lowered nitrogen waste, and (b) a fourth innoculum comprising phosphorous absorbing bacteria to produce a phosphorous enriched bacteria fraction and a phosphorous depleted waste fraction.

This application claims the benefit of U.S. Provisional Application No. 60/145,114, filed Jul. 23, 1999.

### FIELD OF THE INVENTION

The invention relates to the treatment of organic waste.

### BACKGROUND OF THE INVENTION

High efficiency wastewater treatment (e.g., sewage, animal waste and industrial organic waste streams) have become increasingly important as the world's population continues to grow. Wastewater typically has a high water content and requires substantial processing before it can be released because of the contaminants contained within the wastewater.

A wide variety of different wastewater treatment systems have been proposed. A number of wastewater treatment processes comprise “biological” systems utilizing microorganisms contained in an activated biomass, or sludge, for the removal of BOD, phosphorous and/or nitrogen. These treatment processes typically incorporate multiple treatment phases or “zones”, namely: (1) a preliminary treatment area; (2) a primary treatment area; and (3) a secondary treatment area. Preliminary treatment is primarily concerned with the removal of solid inorganics from untreated wastewater. Typically, this preliminary treatment encompasses a two-stage treatment process in which the debris is removed by screens and/or settling. Organic matter is carried out in the fluid stream for subsequent treatment. Primary treatment entails a physical process wherein a portion of the organics, including suspended solids such as feces, food particles, etc. is removed by flotation or sedimentation. Secondary treatment typically encompasses a biological treatment process where microorganisms are utilized to remove remaining organics, nitrogen and phosphorous from the wastewater fluid stream. Microorganism growth and metabolic activity are exploited and controlled through the use of controlled growth conditions.

In large scale applications, biological treatment processes typically utilize a basin or other reservoir in which the wastewater is mixed with a suspension of biomass/sludge. Subsequent growth and metabolism of the microorganisms, and the resultant treatment of the wastewater, is carried out under aerobic and/or anaerobic/anoxic conditions. In most large scale municipal or industrial treatment systems, the various components of the treatment process are performed in discrete basins or reactors. As such, there is a continuous flow of the wastewater from one process step to the next. Biomass containing the active microorganisms may be recycled from one process step to another. The conditioning of such biomass to enhance growth of particularized subgroups of microorganisms possessing a proclivity for performing a specific type of metabolic process (e.g. phosphate removal, nitrogen removal) has been the subject matter of numerous patents, including: U.S. Pat. Nos. 4,056,465; 4,487,697; 4,568,462; 5,344,562. The optimization of other components or aspects of biological wastewater treatment has also engendered a variety of patents, including: U.S. Pat. Nos. 2,788,127; 2,875,151; 3,440,669; 3,543,294; 4,522,722; 4,824,572; 5,290,435; 5,354,471; 5,395,527; 5,480,548; 4,259,182; 4,780,208; 5,252,214; 5,022,993; 5,342,522; 3,957,632; 5,098,572; 5,290,451; Canadian Patent No. 1,064,169; Canadian Patent No. 1,096,976; Canadian Patent No. 1,198,837; Canadian Patent No. 1,304,839; Canadian Patent No. 1,307,059; and Canadian Patent No. 2,041,329.

Biological removal of organic carbon, nitrogen and phosphorus compounds from waste water requires attention to special environmental conditions within the processing equipment. For instance, for bacteria and other microbes to convert organic carbon compounds (measured as BOD) to carbon dioxide and water, a well mixed aerobic environment is required. To convert nitrogen compounds to nitrogen gas and carbon dioxide, nitrosomas and nitrobacter operate in an aerobic environment consuming inorganic carbon. Subsequently, facultative bacteria operate in an anoxic environment consuming organic carbon and liberating nitrogen gas. To biologically tie up phosphate in the cell mass, an anaerobic step to produce volatile fatty acids is required. This is followed by Poly P microbes consuming large amounts of phosphorus required to metabolize the volatile fatty acids in an aerobic environment thus concentrating the phosphate in the biomass (see, e.g., Abstract by Dr. W.Wilson Western Canada Water and Wastewater Conference Calgary AB. Jan 2002.)

The cost of processing commercial, municipal, and agricultural waste in order to reduce or remove BOD, nitrogen and/or and phosphorus stresses the budget of many waste treatment facilities responsible for handling such wastes. A primary cost factor involved in the removal of nitrogen and/or phosphorus from such wastes is the cost of the equipment needed to handle the large volume of wastewater and the amount of energy used to aerate the wastewater.

Accordingly, a need exists for a system capable of reducing the cost to reduce or remove nitrogen and/or phosphorus from commercial, municipal, and agricultural waste.

### SUMMARY OF THE INVENTION

A process for treating waste comprising the steps of (i) adding a first innoculum comprising sulfide-utilizing bacteria to a sulfide-rich organic waste to produce a lowered-sulfide waste, (ii) adding a second innoculum comprising organic digesting bacteria to the lowered-sulfide waste to produce a digested waste, and (iii) adding to the digested waste at least one of (a) a third innoculum comprising bacteria capable of converting a nitrogen-containing compound to N₂ to produce a lowered nitrogen waste, and (b) a fourth innoculum comprising phosphorous absorbing bacteria to produce a phosphorous enriched bacteria fraction and a phosphorous depleted waste fraction.

### DETAILED DESCRIPTION OF THE INVENTION

The invention is a process for treating organic waste containing nitrogen and/or phosphorus which is able to remove or reduce nitrogen and/or phosphorus from the waste at a reduced cost. More particularly, the process reduces the amount of waste requiring treatment to remove nitrogen and/or phosphorus, and decreases the oxygen requirements of the waste in order to achieve good removal of nitrogen and/or phosphorus.

The process is a two-stage process which involves (i) a primary stage comprising treatment of the waste utilizing the process disclosed and described in U.S. Pat. No. 5,958,758 in order to reduce the concentration of biological oxygen demand (BOD), reduce total solids, and reduce total suspended solids, and (ii) a secondary stage comprising treatment of the waste stream from the primary stage to biologically remove or reduce nitrogen and/or phosphorus from the waste stream utilizing any of the widely known biological techniques for achieving such a reduction. The disclosure of U.S. Pat. No. 5,958,758 is hereby incorporated by reference. The two stage process provides a more efficient secondary stage treatment to remove nitrogen and/or phosphorus by allowing smaller treatment vessel(s), smaller air blowers for oxygen needed during the nitrification/denitrification step of the process, and a much reduced processing time for the nutrient removal process. The two-stage process also reduces energy use by requiring less energy to operate the equipment, particularly the aeration blowers. This is particularly true when the secondary stage is a sequenced batch reactor processing system for nitrogen and/or phosphorus removal.

A common sequenced batch reactor processing system for biologically removing or reducing nitrogen and phosphorus from a waste stream utilizes three separate treatment vessels in succession, with each vessel providing certain physical and biological treatment conditions to achieve (i) biological conversion of inorganic nitrogen compounds (e.g., NH₃) to nitrogen gas (N₂) which can be released into the atmosphere, and (ii) biological utilization of phosphorus by microorganisms which incorporate the phosphorus into removable solids which can be removed from the waste stream by a variety of techniques, such as a decanting process.

An alternative batch reactor processing system for biologically removing or reducing nitrogen and phosphorus from a waste stream utilizes a single treatment vessel in which the treatment conditions (e.g., temperature, pressure, pH, biological inoculate, etc.) is changed at selected times during a treatment cycle in order to sequentially achieve the physical and biological treatment conditions necessary to cause (i) biological conversion of inorganic nitrogen to nitrogen gas, and (ii) biological utilization of phosphorus into removable solids.

Primary Stage

The waste is first inoculated with sulfide-utilizing bacteria. The amount of bacteria in the first innoculum will vary with specific treatment conditions, including temperature, pH, and the like. In general, the first innoculum is sufficient to deliver approximately 10⁵-10¹⁰ sulfide-utilizing bacteria per milliliter of waste fluid, and preferably about 10⁶-10⁸ bacteria per milliliter.

The sulfide-utilizing bacterial innoculum can include one or more of the bacteria shown below in Table 1. A preferred innoculum of sulfide-utilizing bacteria is commercially available from Syneco Systems, Inc. (St. Louis Park, Minn.) under the trade name Persnickety Brand 713™.

TABLE 1
SULFIDE-UTILIZING BACTERIA

| Purple | | | Filamentous | Colorless |
|---|---|---|---|---|
| Bacteria | Purple Non-Sulfur | Green Sulfur | Green | Sulfur and |
| Chromatium | Bacteria | Bacteria | Bacteria | Other Bacteria |
| Chromatium | Rhodospirillum | Chlorobium | Chloroflexus | Beggiatoaceae |
| Thiocystis | Rhodobacter | Prosthecochloris | Chloronema | Achromatium |
| Thiospirillum | Rhodopseudomonas | Pelodictyon | Oscillochloris | Thiobacterium |
| Thiocapsa | Rhodomicrobium | Ancalochloris | | Macromonma |
| Lamprocystis | Rhodopila | Chloroherpeton | | Thiospira |
| Lamprobacter | | | | Thiovulum |
| Thiopedia | | | | Bilophocucus |
| | | | | Thiobacillus |
| | | | | Thiomicrospira |
| | | | | Thiodendron |
| | | | | Thiosphaera |
| | | | | Acidiphilium |
| | | | | Thermothrix |
| | | | | Sulfolobus |
| | | | | Acidianus |

The sulfide-utilizing bacteria in the inoculated waste is permitted to acclimate and degrade sulfide compounds present in the waste. After a period of time sufficient to lower sulfide content and provide conditions for efficient growth and activity of organic digesting bacteria a second innoculum of organic digesting bacteria is added. The second innoculum is preferably added when filtered sulfide content is less than 1 ppm, which is typically about 7 to 21 days after addition of the first innoculum. The second innoculum includes one or more species of Bacillus, Desulfotomaculum, Clostridium, and Deimococcaeceae. Species of Bacillus are preferred. Most preferred is a consortium of organic digesting bacteria which is capable of digesting or utilizing a variety of organic compounds. Also preferred are those organic digesting bacteria that produce lytic enzymes capable of degrading organic compounds such as commonly found in animal waste. As with the first innoculum, the amount of bacteria in the second innoculum will vary with specific treatment conditions, including temperature, pH, and the like.

The second innoculum can optionally include lytic enzymes for digesting organic matter. Preferred lytic enzymes include proteases, lipases, cellulases, and amylases.

A most preferred second innoculum contains an equal mixture of three species of Bacillus: licheniformis, amyloliquidifaciens, and subtilis together with protease (10,000 PV units/g), amylase (300 DV units/g), lipase (1500 TAU units/g) and cellulase and is commercially available from Athea Labs, (Milwaukee, Wis.) under the trade name Digestase 900™.

The waste inoculated with the second innoculum is preferred continuously circulated in order to maximize the effect of the bacteria and enzymes. Circulation may be achieved by a number of known methods, including paddle, blade or fluid systems. A most preferred circulation system is a diffused air system placed in, and preferably at the bottom of the waste holding facility to produce a plume of air which rises to the surface to create a mixing action. Suitable air diffusion systems are commercially available, for example, from Clean Flow Laboratories (Plymouth, Minn.). An aspirator-type system which causes circulation by injecting air at the surface of the facility is commercially available from Aeration Industries (Minneapolis, Minn.).

The primary stage is effective for achieving a significant reduction in BOD and thereby a reduction in BOD/TKN (Total Kjeldahl Nitrogen). Such a reduction in BOD significantly reduces equipment and energy demands for completion of the secondary stage. Generally, the higher the BOD/TKN ratio of the waste stream entering the secondary stage, the slower the nitrification rate due to blinding of the nitrification process by excess carbon (e.g., the recommended minimum Dissolved Oxygen (DO) level for effective nitrification is 2.0 mg/l). BOD reduction and nitrification compete for available oxygen within the waste, with BOD reduction typically dominating utilization of available oxygen. This competition for oxygen can significantly increase nitrification processing time for waste streams overloaded with BOD. Without intending to be limited to any particular theory, I believe that the two stage process disclosed herein is effective for significantly increasing nitrification rates with reduced energy utilization because the BOD/TKN ratio of the waste stream subjected to the nitrification process is significantly lowered during the primary stage.

Secondary Stage

The waste stream from the primary stage is treated in a secondary stage to remove phosphorus (P) and/or nitrogen (N). The biological conversion of complex or insoluble compounds containing phosphorus (P) and/or nitrogen (N) typically requires a series of biochemical reactions carried out by several different microbial consortia. These organisms grow under different conditions, have substantially different growth rates, and therefore compete differently for substrates, carbon and energy sources.

Both phosphorus and nitrogenous compounds are encountered in wastewater in two general forms-inorganic and organic—which together make up total phosphorus (TP) or total nitrogen (TN). Complex phosphorus and nitrogen compounds are found in soluble and insoluble states, and usually need to be converted into simple form such as orthophosphate or ammonia before use by most microorganisms.

Facultative anaerobic processes are found to be most effective for converting complex P or N into orthophosphate or ammonia. These processes involves various hydrolyzing enzymes from acclimatized microorganisms.

After biological conversion, most of the N and P are in solution, and only a portion is assimilated into the biomass. The ultimate goal is to reduce the N and P compounds from the water and wastewater to specified levels to meet discharge or reuse requirements. Several biological processes for removal of the N or P compounds are well known in the art. Such conventional systems typically involve suspended growth systems or sludge wasting methods.

To biologically remove soluble P, there is need for a selection system that allows for growth and retention of the P-removal microbial consortia in the bioreactor system in a reasonable concentration. This biomass may then absorb the PO₄-P in relatively high concentrations in its microbial cells. After reaching the maximum capacity under favorable conditions, the biomass is typically removed from the system and disposed of as waste sludge before it can release the absorbed P into the solution again.

The chemistry of nitrogen is more complex because N can exist in seven oxidation states. Many species of bacteria are able to change the oxidation states of N. Nitrogen is commonly removed by inoculating the waste with microbes capable of converting ammonia into nitrogen oxides and converting nitrogen oxides into nitrogen gas.

I claim:

1. A process for treating waste comprising:
(a) adding a first innoculum comprising sulfide-utilizing bacteria to a sulfide-rich organic waste containing a concentration of at least one contaminant selected from nitrogen and phosphorous, to produce a lowered-sulfide waste,
(b) adding a second innoculum comprising organic digesting bacteria to the lowered-sulfide waste to produce a digested waste, and
(c) adding to the digested waste at least one of:
(1) a third innoculum comprising bacteria capable of converting a nitrogen-containing compound to N₂ to produce a lowered nitrogen waste, and
(2) a fourth innoculum comprising phosphorous absorbing bacteria to produce a phosphorous enriched bacteria fraction and a phosphorous depleted waste fraction.

2. The process of claim 1 wherein the organic waste is animal waste.

3. The process of claim 1 wherein the first innoculum includes one or more of Chromatium, Rhodospirillum, Chlorobium, Chloroflexus and Thiobacillus.

4. The process of claim 1 wherein the second innoculum includes one or more of Bacillus, Desulfotomaculum, Clostridium, and Deimococcaeceae

5. The process of claim 4 wherein the second innoculum includes one or more species of Bacillus.

6. The process of claim 5 wherein the second innoculum includes a mixture of .licheniformis, amyloliquidifaciens, and subtilis.

7. A process for treating waste comprising:
(a) adding a first innoculum comprising sulfide-utilizing bacteria to a sulfide-rich and nitrogen-rich organic waste, to produce a lowered-sulfide waste, (b) adding a second innoculum comprising organic digesting bacteria to the lowered-sulfide waste to produce a digested waste, and (c) adding a third innoculum comprising bacteria capable of converting a nitrogen-containing compound to N₂, to the digested waste to produce a lowered nitrogen waste.

8. The process of claim 7 wherein the organic waste is animal waste.

9. The process of claim 7 wherein the first innoculum includes one or more of Chromatium, Rhodospirillum, Chlorobium, Chloroflexus and Thiobacillus.

10. The process of claim 7 wherein the second innoculum includes one or more of Bacillus, Desulfotomaculum, Clostridium, and Deimococcaeceae

11. The process of claim 10 wherein the second innoculum includes one or more species of Bacillus.

12. The process of claim 11 wherein the second innoculum includes a mixture of .licheniformis, amyloliquidifaciens, and subtilis.

13. A process for treating waste comprising:
(a) adding a first innoculum comprising sulfide-utilizing bacteria to a sulfide-rich and phosphorous-rich organic waste, to produce a lowered-sulfide waste,
(b) adding a second innoculum comprising organic digesting bacteria to the lowered-sulfide sulfide waste to produce a digested waste, and
(c) adding a third innoculum comprising phosphorous absorbing bacteria to the digested waste to produce a phosphorous enriched bacteria fraction and a phosphorous depleted waste fraction.

14. The process of claim 13 wherein the organic waste is animal waste.

15. The process of claim 13 wherein the first innoculum includes one or more of Chromatium, Rhodospirillum, Chlorobium, Chloroflexus and Thiobacillus.

16. The process of claim 13 wherein the second innoculum includes one or more of Bacillus, Desulfotomaculum, Clostridium, and Deimococcaeceae

17. The process of claim 16 wherein the second innoculum includes one or more species of Bacillus.

18. The process of claim 17 wherein the second innoculum includes a mixture of licheniformis, amyloliquidifaciens, and subtilis.
